(19) United States
(12) Patent Application Publication
(10) Pub. No.: US 2018/0140457 A1
(43) Pub. Date: May 24, 2018

(54) SYSTEM AND METHOD FOR SURGICAL SUPPORT MAT

(71) Applicant: The General Hospital Corporation, Boston, MA (US)

(72) Inventor: Jaydev SARMA, Walpole, MA (US)

(21) Appl. No.: 15/575,797

(22) PCT Filed: May 20, 2016
(86) PCT No.: PCT/US16/33464

§ 371 Date: Nov. 20, 2017

Related U.S. Application Data
(60) Provisional application No. 62/165,364, filed on May 22, 2015.

(51) Int. Cl. A61F 5/37; A61G 13/12

(52) U.S. Cl. CPC A61F 5/3776; A61G 2200/325; A61G 13/1235

(57) ABSTRACT

A device to secure a patient during a surgery on an operating table. The device includes: a central cushioning mat to be positioned between the patient and the operating table when the patient is placed on the device. The device includes a first flexible side mat attached to the central cushioning mat and configured to be folded around a first arm of the patient. The device also includes a second flexible side mat attached to the central cushioning mat and configured to be folded around a second arm of the patient. The device further includes a locking mechanism configured to hold the first and second flexible side mats in place during the surgery.

### CROSS-REFERENCE TO RELATED APPLICATIONS

This application is based on, claims priority to, and incorporates herein by reference U.S. Provisional Patent Application Ser. No. 62/165,364 filed on May 22, 2015.

### STATEMENT REGARDING FEDERALLY SPONSORED RESEARCH

N/A

### BACKGROUND OF THE INVENTION

The present application is directed to a device for securing a patient and, more particularly, a device for securing a patient's arms during surgical procedures.

Positioning injuries of the upper extremities resulting from aggressive tucking of the patient's arms under drapes has resulted in the development of compartment syndrome, and nerve and tissue damage in patients. The common habit of taking the drape from under the patient and enveloping the arm in its entirety can result in cutting off of the circulation to the arm, venous stasis and, in some cases, nerve entrapment. It can also stop the flow of intravenous solutions, which can lead to extravasations of intravenous fluids that can go unnoticed under the drapes.

Therefore, it would be desirable to provide systems and methods that facilitate fast and easy securing of a patient on an operating table in a surgery without the risk of causing positioning injuries of the upper extremities.

### SUMMARY OF THE INVENTION

The present invention overcomes the aforementioned drawbacks by providing a device to secure a patient on an operating table. In a first aspect, a system is provided for restraining a patient during a surgical procedure performed on a subject positioned on an operating table. The system includes a central mat configured to extend from a medial to a lateral portion of the subject to align a first lateral periphery and a second lateral periphery of the central mat to extend from respective superior to inferior lateral sides of the subject. The system further includes a first restraining flange and a second restraining flange. The first restraining flange extends from the first lateral periphery of the central mat to wrap around a first arm of the subject when extending along the first lateral side of the subject. The second restraining flange extends from the second lateral periphery of the central mat to wrap around a second arm of the subject when extending along the second lateral side of the subject. The system further includes a locking system configured to selectively engage the first restraining flange to maintain the first restraining flange wrapped about the first arm of the subject and to selectively engage the second restraining flange to maintain the second restraining flange wrapped about the second arm of the subject.

In a second aspect, a device is provided. The device includes a central cushioning mat to be positioned between the patient and the operating table when the patient is placed on the device. The device includes a first flexible side mat attached to the central cushioning mat and configured to be folded around a first arm of the patient. The device also includes a second flexible side mat attached to the central cushioning mat and configured to be folded around a second arm of the patient. The device further includes a locking mechanism configured to hold the first and second flexible side mats in place during the surgery.

In a third aspect, a method is provided for securing a patient on a patient table. The method may include: providing a device including: a central cushioning mat, a locking mechanism, and a first and second flexible side mat attached to opposite sides of the central cushioning mat. The central cushioning mat is positioned under the patient and above the operating table. The first flexible side mat is folded around a first arm of the patient. The second flexible side mat is folded around a second arm of the patient. The locking mechanism then holds the first flexible side mat and the second flexible side mat with the first and second arms in place during the surgery.

These and other features and advantages of the present invention will become apparent upon reading the following detailed description when taken in conjunction with the drawings.

### BRIEF DESCRIPTION OF THE DRAWINGS

FIG. 1A is a perspective view of a patient and patient positioning system in accordance with one or more embodiments of the disclosure;

FIG. 1B is another perspective view of the patient and patient positioning system of FIG. 1;

FIG. 2 is another perspective view of the patient positioning device in a non-working or non-operational state;

FIG. 3 is a bottom, plan view of the patient positioning device in a non-working state of FIG. 2;

FIG. 4 is a top view of the patient positioning device in a working state;

FIG. 5 is aside view of the patient positioning device when one of the side mat is lifted to show a part of the bottom of the side mat;

FIG. 6 is a top plan view of a patient positioning device according to one or more embodiments of the disclosure;

FIG. 7 is a side view of the patient positioning device in a folded state of FIG. 6; and

FIG. 8 is a flow chart illustrating an example method according to one or more embodiments of the disclosure.

### DETAILED DESCRIPTION OF THE INVENTION

The common habit of taking the drape of sheets from under the patient and enveloping the arm in its entirety can result in cutting off of the circulation to the arm, venous stasis and, in some cases, nerve entrapment. It may also stop the flow of intravenous solutions, which lead to extravasations of intravenous fluids that can go unnoticed under the drapes.

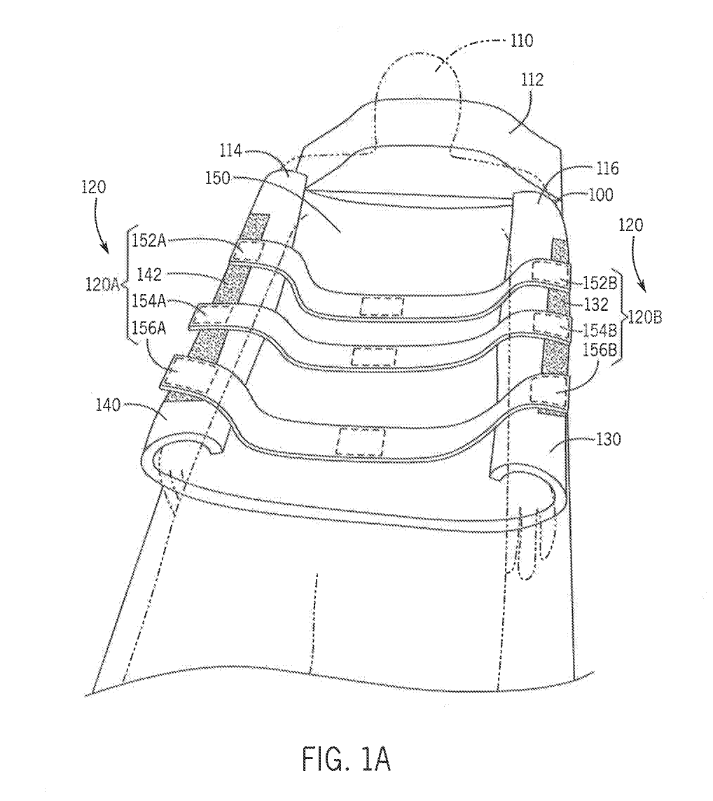

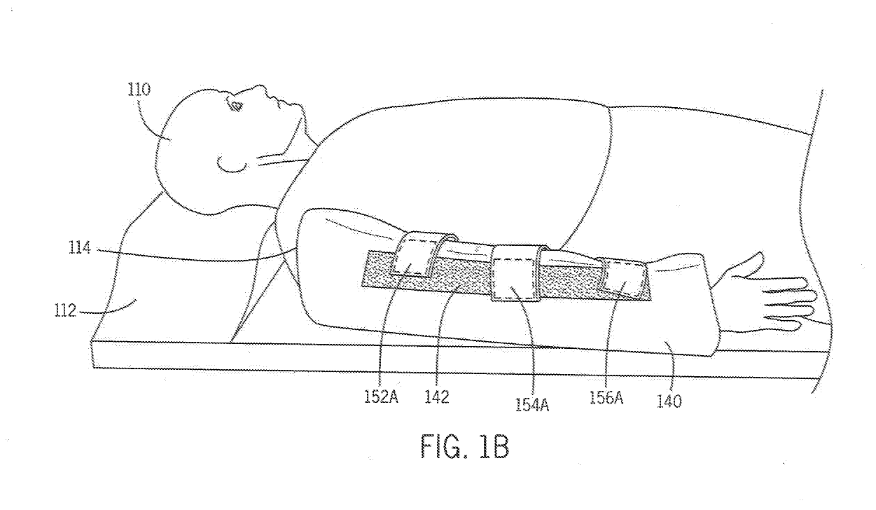

This disclosure provides a patient positioning system to overcome these and other challenges. FIG. 1A is a perspective view of a patient 110 and an example patient positioning system 100 in accordance with one or more embodiments of the disclosure. The example patient positioning system 100 secure the subject 110 on a patient table 112. The patient positioning system 100 includes a central mat 150 configured to extend from a medial to a lateral portion of the subject 110 to align a first lateral periphery and a second lateral periphery of the central mat to extend from respective superior to inferior lateral sides of the subject 110. As shown in FIG. 3, the first restraining flange 130 extends from the first lateral periphery 157 of the central mat 150 and the second restraining flange 140 extends from the second lateral periphery 158 of the central mat 150.

Here, the patient positioning system 100 secures the arms 114 and 116 of the subject 110 in a desired position without the risk of positioning injuries. The left arm 114 is secured by a first restraining flange 130 that wraps around the arm 114 of the subject 110 when extending along the first lateral side of the subject 110.

FIG. 1B is a side view of the patient and patient positioning system of FIG. 1. In FIG. 1B, the right arm 116 is secured by a second restraining flange 140 that wraps around the arm 116 of the subject 110 when extending along the first lateral side of the subject 110.

As shown in FIGS. 1A and 1B, the patient subject 110 lies on a central mat 150 of the positioning system 100, which is positioned between the patient subject 110 and the patient table 112. The positioning system 100 includes a locking system 120 that includes a first locking sub-system 120A and a second locking sub-system 120B. The first sub-system 120A is configured to selectively engage the first restraining flange 140 to maintain the first restraining flange 140 wrapped about the first arm 114 of the subject 110. The second sub-system 120B configured to selectively engage the second restraining flange 130 to maintain the second restraining flange 130 wrapped about the second arm 116 of the subject 110.

The first sub-system 120A includes strap ends 152A, 154A, and 156A. The strap ends may include touch fasteners or other fasteners on each of them. Though three straps are illustrated, more or less straps may be utilized. Likewise, instead of multiple strap, a continuous sheet or other locking mechanisms may be utilized. In the illustrated configuration, the first sub-system 120A further includes a lineal fabric strip 142 attached to the first restraining flange 140. As shown in FIG. 3, the lineal fabric strip 142 is attached to a bottom side 140B of the first flexible side mat 140. The lineal fabric strip 142 includes corresponding touch fasteners so that the strap ends 152A, 154A, and 156A can be stuck to the lineal fabric strip 142 and thus lock the first restraining flange 140 and the patient arm a desired position.

Similarly, the second sub-system 120B includes strap ends 152B, 154B, and 156B, which may include touch fasteners or other fasteners on each of them. Again, though three straps are illustrated, more or less straps may be utilized. Likewise, instead of multiple strap, a continuous sheet or other locking mechanisms may be utilized. In the illustrated configuration, the second sub-system 120B further includes a lineal fabric strip 132 attached to the second restraining flange 130. As shown in FIG. 3, the lineal fabric strip 132 is attached to a bottom side 130B of the second flexible side mat 130. The lineal fabric strip 132 includes corresponding touch fasteners so that the strap ends 152B, 154B, and 156B can be stuck to the lineal fabric strip 132 and thus lock the second restraining flange 130 and the patient arm a desired position.

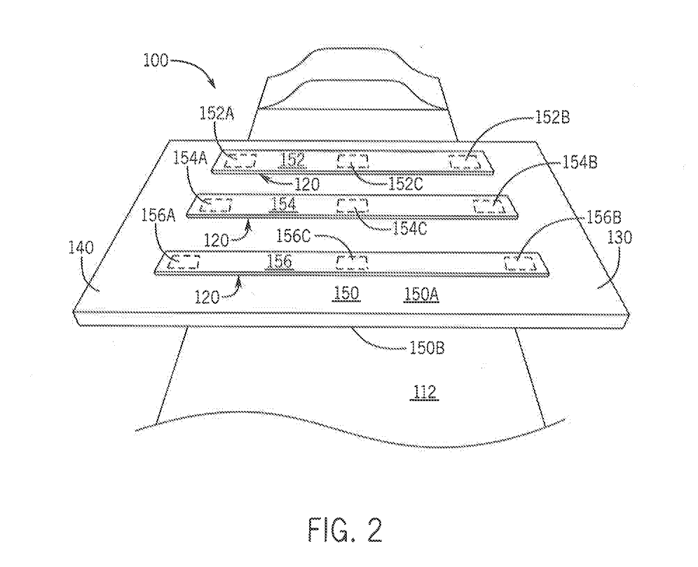

FIG. 2 is a top view of the patient positioning device in a non-working state. The central mat 150 includes an upper or superior surface 150A and a lower or inferior surface 150B. The patient positioning device 100 is designed to be disposed on the patient table 112 such that the restraining flanges 130, 140 extend beyond the table 112. In this way, when a patient subject lies on the central mat 150, the straps 152, 154, and 156 can extend from the medial to the lateral portion of the subject to be coupled with the first restraining flange 140 and the second restraining flange 130 around the subject patient's arms.

In FIG. 2, the first strap 152 includes a first end 152A, a second end 152B, and a middle portion 152C between the first end 152A and the second end 152B. The first end 152A and the second end 152B both include touch fasteners so that they can be attached to the central mat 150 in the non-working state. For example, the touch fasteners may include respective loop or hook patches and the central mat 150 includes reciprocal loop or hook patches configured to engage and interlock with the respective loop or hook patches on the first end 152A and the second end 152B. The central mat 150 may include one or more slots to accommodate the first strap 152 so that the strap 152 may be moved by the user. Further, the first strap 152 may be cleaned or replaced if necessary.

Similarly, the second strap 154 also includes a first end 154A, a second end 154B, and a middle portion 154C between the first end 154A and the second end 154B. The first end 154A and the second end 154B both include touch fasteners so that they can be attached to the central mat 150 in the non-working state. The third strap 156 also includes a first end 156A, a second end 156B, and a middle portion 156C between the first end 156A and the second end 156B. The first end 156A and the second end 154B both include touch fasteners so that they can be attached to the central mat 150 in the non-working state. Although the example positioning system 100 in FIG. 2 includes three straps, a person of ordinary skill in the art would understand that more straps may be added if needed. Further, the positioning system 100 may only need two straps if the patient subject has a relatively small size.

The central mat 150 may include one or more slots to accommodate the second strap 154 so that the second strap 154 may be moved, removed, or replaced by the user. Further, the central mat 150 may include one or more slots to accommodate the third strap 156 so that the second strap 154 may be moved, removed, or replaced by the user. Alternatively or additionally, the central mat 150 may include touch fasteners or other fasteners to temporarily fix the position of the straps 152, 154, and 156 by attaching the middle portions 152C, 154C, and 156C of the straps. If needed, one of the straps may include an embedded sensor that measures the blood pressure of the patient subject. The embedded may be used to measure other biological signals of the patient subject.

As shown in FIG. 2, the side mats 130 and 140 are in a flat state in the non-working state. While in FIGS. 1A and 16, the side mats 130 and 140 are bent to become the restraining flanges in a curved state.

FIG. 3 is a bottom view of the patient positioning device in a non-working state of FIG. 2. In FIG. 3, the patient positioning device 100 includes a central mat 150 having a bottom surface 150B, which is usually in direct contact with a patient bed in a working state. The central mat 150 includes a first lateral periphery 157 and a second lateral periphery 158. The first lateral periphery 157 may include a sewing line or other boundary lines. Similarly the second lateral periphery 158 may include a sewing line or other boundary lines. The straps 152, 154, and 156 are shown in dashed boxes because they are attached to the upper surface of the central mat 150 and cannot be seen from this angle. The patient positioning device 100 may have a thickness of 1 to 3 inches, or more preferably in the range of 1.0 to 1.5 inches.

The patient positioning device 100 has a length L and a width K. The length L may be in the range of 15 to 30 inches, or more preferably in the range of 20 to 25 inches. The width K may be in the range of 28 to 40 inches, or more preferably in the range of 30 to 36 inches. The central mat 150 has a length L and a width K4. The width K4 may be in the range of 24 inches to 30 inches. The straps 152, 154, and 156 have a length L2 and a width K2. The length L2 may be in the range of 1.5 inches to 4.5 inches, or more preferably in the range of 2 to 3 inches. The width K2 may be in the range of 20 inches to 30 inches, or more preferably in the range of 23 to 27 inches.

In FIG. 3, the first side mat 130 includes a lineal strap 132 on its bottom surface 130B. The second side mat 140 includes a second lineal strap 142 on its bottom surface 140B. Both lineal straps 132 and 142 may include touch fasteners or other fasteners to lock the straps 152, 154, and 156 so that the side mats 130 and 140 lock the patient arms in a desired position. The first side mat 130 may have a length L and a width K3. The second side mat 140 have a length L and a width K3 as well. The width K3 may be in the range of 4 to 12 inches, or more preferably in the range of 6 to 10 inches. The lineal straps 132 and 142 may each have a length of L1 and a width K1. The length L1 may be in the range of 10 inches to 20 inches, or more preferably in the range of 12 to 18 inches. The width K1 may be in the range of 1.5 inch to 6 inches, or more preferably in the range of 2 to 4 inches.

For example, the first lateral periphery 157 may include a zip fastener or other fasteners to attach the first side mat 130. In this case, the user may select different side mats with a preferred dimension to accommodate the different arm sizes of the patient subject. Similarly, the second lateral periphery 158 may also include a zip fastener or other fasteners to attach the second side mat 140.

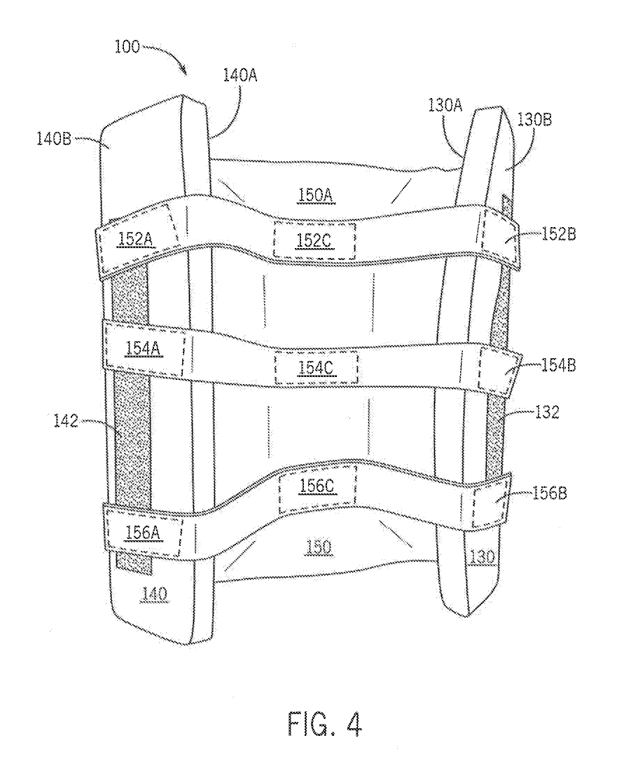

FIG. 4 is a top view of the patient positioning device in a working state. In the working state, the straps ends 152A, 154A, and 156A are attached to the lineal strap 142 on the surface 140B. The straps ends 152B, 154B, and 156B are attached to the lineal strap 132 on the surface 130B. Here, the middle portions 152C, 154C, and 156C are stitched to the upper surface 150A of the central mat 150.

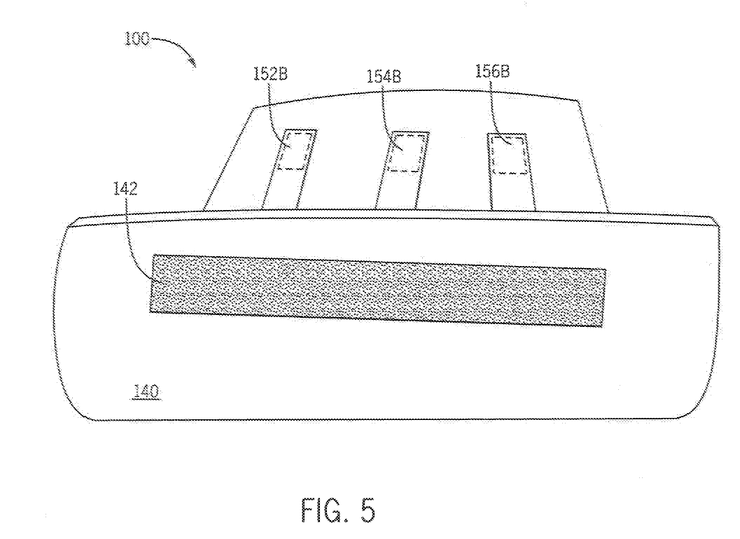

FIG. 5 is aside view of the patient positioning device when one of the side mat is lifted to show a part of the bottom of the side mat. In FIG. 5, the lineal strap 142 is stitched to the side mat 140. A person having ordinary skill in the art understands that the lineal strap 142 may be attached to the side mat 140 using other locking mechanisms such as zippers, buttons, and etc.

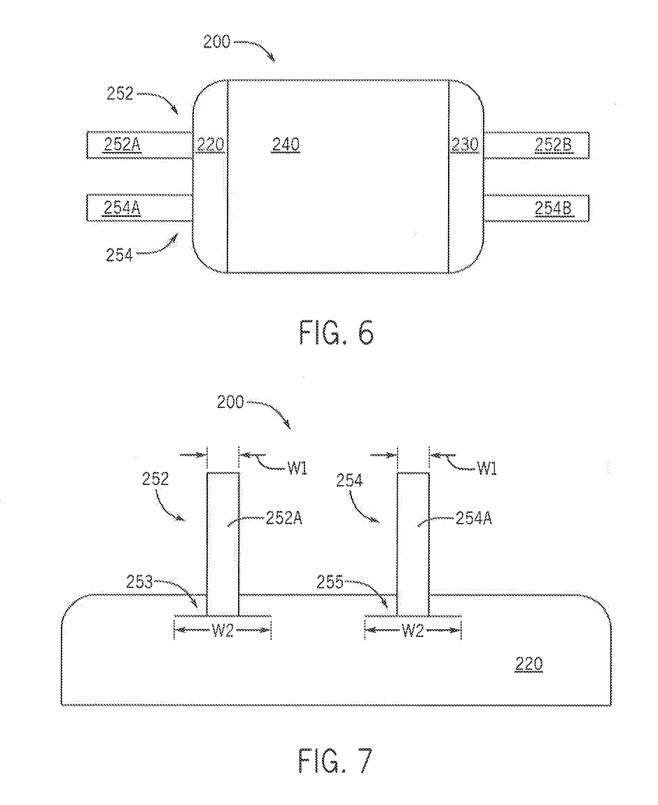

FIG. 6 is a top view of a patient positioning device according to one or more embodiments of the disclosure. The patient positioning device 200 includes a central mat 240, a first side mat 220, and a second side mat 230. The central mat 240 is in the middle between the first side mat 220 and the second side mat 230.

For example, the central mat 240 may include a rectangle shape having a width of 20 inches and a length of 30 inches. There first side mat 220 and the second &de mat 230 may include two foldable side (arm) pieces attached to the central mat. The side mats may have a width of 6 inches and a length of 30 inches. The patient positioning device 200 may be positioned to be flush with the upper margin of the shoulder joints of a patient subject. The side mats 220 and 230 can be folded over the arms so that the arms would lie comfortably inside.

The patient positioning device 200 may include two sets of adjustable straps coming from the side mats 220 and 230. The two adjustable straps 252 and 254 may attach to the outer surface of the mat and may be tucked under the patient's body and hold the side mats 220 and 230 in place during surgery. The first adjustable strap 252 includes a first end 252A and, a second end 252B. The second adjustable strap 254 includes a first end 254A and a second end 254B. The strap ends 252A, 252B, 254A, and 254B may include touch fasteners on each one of them.

For example, the central mat 240 may include a non slip gel mat. The central mat 240 and the two side mats 220 and 230 may be laid on the operating table mattress before positioning the patient on the operating table. The patient positioning device 200 is positioned such that the upper edge of the central mat 240 is at the level of the shoulder joints and with the patient on this central mat 240 with his/her arms by his/her side. The side mats 220 and 230 may then be lifted up and the straps 252 and 254 can be tucked under the patient's body. The arms with any lines can then be resting in a neutral position by the side of the patient and would be protected on its outer surface by the side mats 220 and 230, which may include gel or other similar material in them.

FIG. 7 is a side view of the patient positioning device in a folded state of FIG. 6. For example, the side mat 220 has a length of 30 inches and a width of 6 inches. Two slots 253 and 255 are provided in the side mat 220. The slots 253 and 255 may have a width W2 of six inches. The two straps 252 and 254 may have a width W1 of 1.5 inches and a length of 10 inches. The two straps 252 and 254 can be fed through these slots 253 and 255. The slots may be slid into the desired position to suit each patient. These straps 252 and 254 can be tucked under the patient and hold the arm piece in place during surgery. The straps 252 and 254 can slide over the two six-inch slots 253 and 255 and can allow the patient positioning device 200 to be used for patients of all sizes and shapes.

As shown in FIG. 7, the straps would be attached to the outer surface of the arm piece by means of loops in two six-inch slits 253 and 255, thus allowing them to be positioned where needed. This enables the straps 252 and 254 to be adjusted for patients of different sizes, shapes and heights. It can also allow for the inspection of intravenous (IV) sites during the operative procedure if needed.

The arms tucked position is used in head and neck, Oromaxillofacial, and laparoscopic operations. This device can enable the positioning of the patient in a quick and safe manner.

FIG. 8 is a flow chart illustrating an example method according to one or more embodiments of the disclosure. In FIG. 8, the example method 300 includes one or more of the following acts.

In act 310, a device is provided. The device includes: a central cushioning mat, a locking mechanism, and a first and second flexible side mat attached to opposite sides of the central cushioning mat. One example of the device is described above. The locking mechanism may include a first lineal fabric strip attached to a bottom side of the first flexible side mat. The locking mechanism may further include a second lineal fabric strip attached to a bottom side of the second flexible side mat.

In act 320, the central cushioning mat is positioned under the patient and above the operating table. The central cushioning mat may have a size to be consistent with the size of the patient subject.

In act 330, the first flexible side mat is folded around a first arm of the patient. In act 340, the second flexible side mat is folded around a second arm of the patient. The side mats may include material that can be used to lock the shape of the side mats. For example, the side mats may include touch fasteners or other types of fasteners.

In act 350, the locking mechanism holds the first flexible side mat and the second flexible side mat with the first and second arms in place during the surgery. The locking mechanism may use touch fasteners or other types of fasteners to hold the flexible side mats and the arms in place. The locking mechanism further includes an adjustable strap attached to the central cushioning mat, the adjustable strap including a third lineal fabric strip. In act 360, the adjustable strap may be fastened to the first lineal fabric strip and the second lineal fabric strip by touch fasteners disposed on the first, second, and third lineal fabric strips.

Thus, the above-described invention provides a device to secure a patient in a selected body position on an operating table. The device can include a central cushioning mat, a locking mechanism, and a first and second flexible side mat attached to opposite sides of the central cushioning mat. The central cushioning mat may be positioned between the patient and the operating table when the patient is placed on the device. The first flexible side mat is attached to the central cushioning mat and configured to be folded around a first arm of the patient. The second flexible side mat is attached to the central cushioning mat and configured to be folded around a second arm of the patient. The locking mechanism is configured to hold the first and second flexible side mats in place during the surgery.

The present invention has been described in terms of one or more preferred embodiments, and it should be appreciated that many equivalents, alternatives, variations, and modifications, aside from those expressly stated, are possible and within the scope of the invention.

We claim:

1. A system for restraining a patient during a surgical procedure performed on a subject positioned on an operating table, the system comprising:
a central mat configured to extend from a medial to a lateral portion of the subject to align a first lateral periphery and a second lateral periphery of the central mat to extend from respective superior to inferior lateral sides of the subject;
a first restraining flange extending from the first lateral periphery of the central mat to wrap around a first arm of the subject when extending along the first lateral side of the subject;
a second restraining flange extending from the second lateral periphery of the central mat to wrap around a second arm of the subject when extending along the second lateral side of the subject; and
a locking system configured to selectively engage the first restraining flange to maintain the first restraining flange wrapped about the first arm of the subject and to selectively engage the second restraining flange to maintain the second restraining flange wrapped about the second arm of the subject.

2. The system of claim 1, wherein the locking system includes a plurality of straps that are coupled to the central mat and extend from the medial to the lateral portion of the subject to be coupled with the first restraining flange and the second restraining flange.

3. The system of claim 2, wherein the first restraining flange and the second restraining flange include respective loop or hook patches extending thereon and wherein the plurality of straps include reciprocal loop or hook patches configured to engage and interlock with the respective loop or hook patches extending along the first restraining flange and the second restraining flange to maintain the first restraining flange wrapped about the first arm of the subject and maintain the second restraining flange wrapped about the second arm of the subject.

4. The system of claim 1 wherein the central mat includes a cushion configured to receive a patient thereon in a prone position or a repose position.

5. A device to secure a patient during a surgery on an operating table, the device comprising:
a central cushioning mat to be positioned between the patient and the operating table when the patient is placed on the device;
a first flexible side mat attached to the central cushioning mat and configured to be folded around a first arm of the patient;
a second flexible side mat attached to the central cushioning mat and configured to be folded around a second arm of the patient; and
a locking mechanism configured to hold the first and second flexible side mats in place during the surgery.

6. The device of claim 5, wherein the locking mechanism comprises a first lineal fabric strip attached to a bottom side of the first flexible side mat.

7. The device of claim 6, wherein the locking mechanism further comprises a second lineal fabric strip attached to a bottom side of the second flexible side mat.

8. The device of claim 7, wherein the locking mechanism further comprises an adjustable strap attached to the central cushioning mat.

9. The device of claim 8, wherein the adjustable strap comprises a third lineal fabric strip attached to an end of the adjustable strap, and wherein the first, second, and third lineal fabric strips include touch fasteners.

10. The device of claim 9, wherein the first lineal fabric strip and the second lineal fabric strip are parallel to each other.

11. The device of claim 9, wherein the adjustable strap is perpendicular to the first lineal fabric strap when the first flexible side mat is not folded.

12. The device of claim 9, wherein the adjustable strap is perpendicular to the second lineal fabric strap when the second flexible side mat is not folded.

13. The device of claim 9, wherein the first lineal fabric strip and the third lineal fabric strip fasten together to hold the first arm of the patient in place during the surgery.

14. The device of claim 9, wherein the second lineal fabric strip and the third lineal fabric strip fasten together to hold the second arm of the patient in place during the surgery.

15. A method for securing a patient during a surgery on an operating table, the method comprising:
providing a device comprising: a central cushioning mat, a locking mechanism, and a first and second flexible side mat attached to opposite sides of the central cushioning mat;
positioning the central cushioning mat under the patient and above the operating table;
folding the first flexible side mat around a first arm of the patient;
folding the second flexible side mat around a second arm of the patient; and
holding, by the locking mechanism, the first flexible side mat and the second flexible side mat with the first and second arms in place during the surgery.

16. The method of claim 15, wherein the locking mechanism comprises a first lineal fabric strip attached to a bottom side of the first flexible side mat.

17. The method of claim 16, wherein the locking mechanism further comprises a second lineal fabric strip attached to a bottom side of the second flexible side mat.

18. The method of claim 17, wherein the locking mechanism further comprises an adjustable strap attached to the central cushioning mat, the adjustable strap including a third lineal fabric strip.

19. The method of claim 18, further comprising:
fastening the adjustable strap to the first lineal fabric strip and the second lineal fabric strip by touch fasteners disposed on the first, second, and third lineal fabric strips.
